(19) United States
(12) Patent Application Publication
(10) Pub. No.: US 2006/0100601 A1
(43) Pub. Date: May 11, 2006

(54) Disposable absorbent garment with elastomeric side panels

(75) Inventor: Susan O'Connell, Coronado, CA (US)

Correspondence Address: WOOD, PHILLIPS, KATZ, CLARK & MORTIMER, 500 W. MADISON STREET, SUITE 3800, CHICAGO, IL, 60661, US

(73) Assignee: Productos Internacionales Mabe S.A. de CV

(21) Appl. No.: 10/984,168

(22) Filed: Nov. 8, 2004

(51) Int. Cl. A61F 13/15

(52) U.S. Cl. 604/389

(57) ABSTRACT

A disposable absorbent garment includes an absorbent assembly, and a pair of elastomeric side panels configured to facilitate fitment of the garment to a wearer. Notably, each elastomeric side panel comprises an elastomeric substrate having a proximal edge, a distal edge, and first and second connecting edges extending between the proximal and distal edges. In accordance with the present invention, the proximal and distal edges of each elastomeric side panel are linear and parallel to each other, while the first and second connecting edges are linear and non-parallel, and non-perpendicular to the distal edge. Each of the connecting edges defines an angle, greater than 90°, relative to the distal edge, with one of these angles being greater than the other.

### TECHNICAL FIELD

The present invention relates generally to disposable absorbent garments, such as disposable diapers and adult incontinence products, and more particularly to a disposable absorbent garment having elastomeric side panels which facilitate improved fit of the garment to a wearer for enhanced comfort and containment.

### BACKGROUND OF THE INVENTION

Disposable absorbent garments, such as in the form of disposable diapers, have come into widespread use by virtue of their highly effective containment characteristics, and the convenience with which such products can be used. Disposable absorbent garments may also be configured for use by incontinent adults.

A typical absorbent disposable garment includes an absorbent assembly, typically comprising comminuted wood pulp (wood fluff), and superabsorbent polymer (SAP), or other absorbent materials. The garment further typically includes a fastening arrangement for securing the garment generally about the waist of a wearer. Such fastening arrangements may include pressure-sensitive adhesive tape and/or mechanical fasteners, typically in the form of hook-and-loop cooperating elements.

In order to facilitate the fit of such disposable garments to a wearer, the use of elastomeric materials in such garments has become increasingly common. In addition to the use of such elastomeric components in the leg portions and waistband of such garments, use of elastomeric material in the fastening arrangement of such garments has become more widespread since such arrangements further facilitate effective fit, and comfort for the wearer. By way of background, U.S. Pat. No. 5,399,219, and No. 5,496,298, both hereby incorporated by reference, disclose disposable absorbent garments in the form of disposable diapers, including fastening arrangements comprising elastomeric material.

While use of tab-like fastening arrangements for disposable absorbent garments is known, it has been found that the fit of such a garment can be improved by providing relatively larger elastomeric fastening elements, which effectively form side panels for the disposable absorbent garment. However, it has been found that the shape of such elastomeric side panel elements is very important in providing a comfortable fit for the wearer, while at the same time maintaining the desired tension of the garment about the waist, body, and legs of the wearer. Additionally, it is frequently desirable that a disposable absorbent garment, such as a diaper, be configured such that the garment can be partially or completely removed by merely lowering the garment, without opening or detachment of the fastening system. Presently available disposable absorbent garments including elastomeric side panel components are not believed to adequately provide this versatility in use.

A need therefore exists for an improved disposable absorbent garment having elastomeric side panels, with the side panels configured to facilitate improved fit and comfort for the wearer.

### SUMMARY OF THE INVENTION

A disposable absorbent garment embodying the principles of the present invention includes a pair of elastomeric side panels each generally having a trapezoidal configuration. Notably, each of the elastomeric side panels is non-symmetrical, with the specific configuration in angular relationship of the edges of each side panel configured to facilitate wearer fit and comfort.

In accordance with the illustrated embodiment, the present disposable absorbent garment includes an absorbent assembly having front and rear portions, and opposite marginal portions. In a typical configuration, the absorbent assembly includes an absorbent matrix, typically comprising comminuted wood pulp and superabsorbent polymer, with the absorbent matrix sandwiched between a liquid-permeable topsheet, positionable adjacent the wearer, and a liquid-impermeable backsheet which provides containment for the absorbent assembly.

The present disposable absorbent garment further includes a pair of elastomeric side panels configured to facilitate fitment of the garment to a wearer. Each elastomeric side panel comprises an elastomeric substrate, and an associated fastening element. The fastening element may be provided in the form of a pressure-sensitive adhesive fastening device, or a mechanical fastening device, comprising one of a hook-and-loop fastening arrangement.

The specific configuration of each of the elastomeric side panels has been selected to facilitate wearer fit and comfort. In particular, the elastomeric substrate of each side panel comprises a proximal edge, a distal edge, and first and second connecting edges extending between the proximal and distal edges. The proximal edge is joined to a respective one of the opposite marginal portions of the absorbent assembly at the rear portion thereof, thus generally providing ears at the rear portion of the garment for securing it in place on a wearer. The distal edge of each elastomeric substrate has the respective fastening element joined thereto for securing each elastomeric side panel in overlapping relationship with the front portion of the absorbent assembly.

The proximal edge and the distal edge of each elastomeric substrate are linear, and parallel to each other. In contrast, the first connecting edge and the second connecting edge of each elastomeric substrate are linear and non-parallel to each other, and non-perpendicular to the distal edge of the substrate.

An angle α (alpha) is defined between the first connecting edge and the distal edge of each elastomeric substrate, with angle α being greater than 90°. An angle β (beta) is defined between the second connecting edge and the distal edge of each elastomeric substrate, with angle β being greater than 90°, and greater than angle alpha/α.

In the preferred form, the difference between angle α and angle β is greater than about 15°, and preferably less than about 40°. The sum of angle α and angle β is preferably less than about 260°.

Angle α is preferably greater than about 100°, more preferably greater than about 110°, and more preferably greater than about 120°. Similarly, angle β is preferably greater than about 120°, more preferably greater than about 130°, and more preferably greater than about 140°.

In order to facilitate the desired fit of the present absorbent garment about the waist of a wearer, the proximal edge of each elastomeric substrate has a length between about 10.3 cm and about 20 cm. The proximal edge of each elastomeric substrate comprises about 20%-35% of a length of the disposable absorbent garment. A ratio of the length of the proximal edge to the distal edge of each elastomeric substrate is between about 2:1 and about 8:1.

Other features and advantages of the present invention will become readily apparent from the following detailed description, the accompanying drawings, and the appended claims.

### BRIEF DESCRIPTION OF THE DRAWINGS

FIG. 1 is a top plan view of a disposable absorbent garment, illustrated as a disposable diaper embodying the principles of the present invention;

FIGS. 2 through 8 illustrate various embodiments of elastomeric side panels of the disposable absorbent garment illustrated in FIG. 1;

FIG. 9 is a diagrammatic view of forces generated within an elastomeric side panel of a known disposable absorbent garment construct; and

FIG. 10 is a diagrammatic view of forces generated within an elastomeric side panel of the disposable absorbent garment of the present invention.

### DETAILED DESCRIPTION

While the present invention is susceptible of embodiment in various forms, there is shown in the drawings, and will hereinafter be described, presently preferred embodiments, with the understanding that the present disclosure is to be considered as an exemplification of the invention, and is not intended to limit the invention to the specific embodiments illustrated.

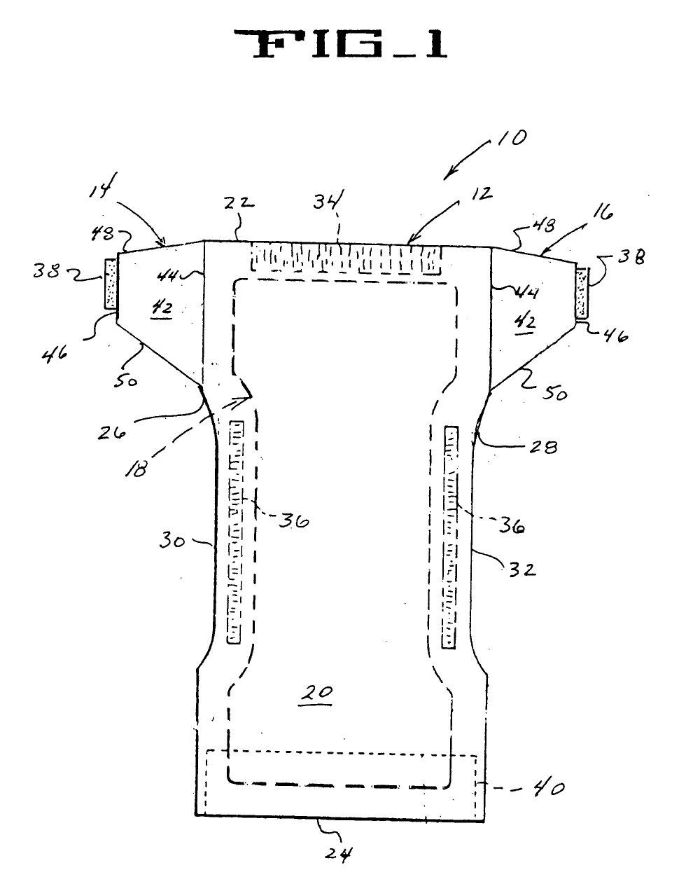

With reference first to FIG. 1, therein is illustrated a disposable absorbent garment illustrated as a disposable diaper 10, embodying the principles of the present invention. As will be understood by those familiar with the art, disposable diaper 10 can be readily configured in a wide variety of sizes so as to accommodate use on infants and children ranging from newborns to toddlers. Diaper 10 can also be readily sized and configured for use by small children. While the present disposable absorbent garment has been illustrated as a disposable diaper, it will be further understood that this type of garment can be readily sized and configured for use by incontinent adults.

Disposable diaper 10 includes an absorbent assembly, generally designated 12, and a pair of elastomeric side panels 14, 16 joined to the absorbent assembly 12. More specifically, absorbent assembly 12 typically comprises an absorbent matrix 18, which may be generally hourglass in shape as illustrated, which ordinarily comprises comminuted wood pulp (wood fluff) and superabsorbent polymer (SAP). The absorbent matrix 18 is ordinarily sandwiched between an associated liquid-permeable topsheet or coversheet 20, positionable adjacent to the wearer of the diaper, and an associated liquid-impermeable backsheet, which typically may be coextensive with the topsheet 20. The absorbent assembly 12 may further include one or more additional layers, such as tissue, so-called liquid distribution or transfer layers; and such other components as are known in the art.

The absorbent assembly 12 includes a rear portion 22, and a front portion 24, and opposite side marginal portions 26 and 28. The opposite marginal portions of the absorbent assembly generally define leg openings at 30, 32, generally at opposite sides of a crotch portion of the garment, with the garment fitting generally between and about the legs of a wearer, with the front and rear portions of the absorbent assembly positioned generally about the waist of the wearer.

In order to facilitate fit and conformance of the disposable diaper 10 to a wearer, the construction may include one or more waist elastic elements 34, and one or more leg elastic elements 36 positioned generally along each of the leg openings of the garment.

In accordance with the present invention, each of elastomeric side panels 14, 16 has been specifically configured to facilitate fit and comfort for the wearer of the disposable absorbent garment. In order to secure the garment in place on a wearer, each of the elastomeric side panels 14, 16 includes a fastening element 38, which may typically comprise a pressure-sensitive adhesive device, or a mechanical fastener provided in the form of one element of a hook-and-loop fastening arrangement. In the illustrated embodiment, each of fastening elements 38 is illustrated in the form of a hook portion of a hook-and-loop fastening mechanism, with the garment further including an exteriorly positioned loop component 40 positioned at the outside surface of the front of the absorbent assembly 12 of the garment. By this arrangement, each of the elastomeric side panels 14, 16 can be secured in overlapping relationship with the front portion of the absorbent assembly, with releasable securement of the hook portion of each fastening element 38 with the loop material 40 positioned on the outside surface of the front portion of the absorbent assembly.

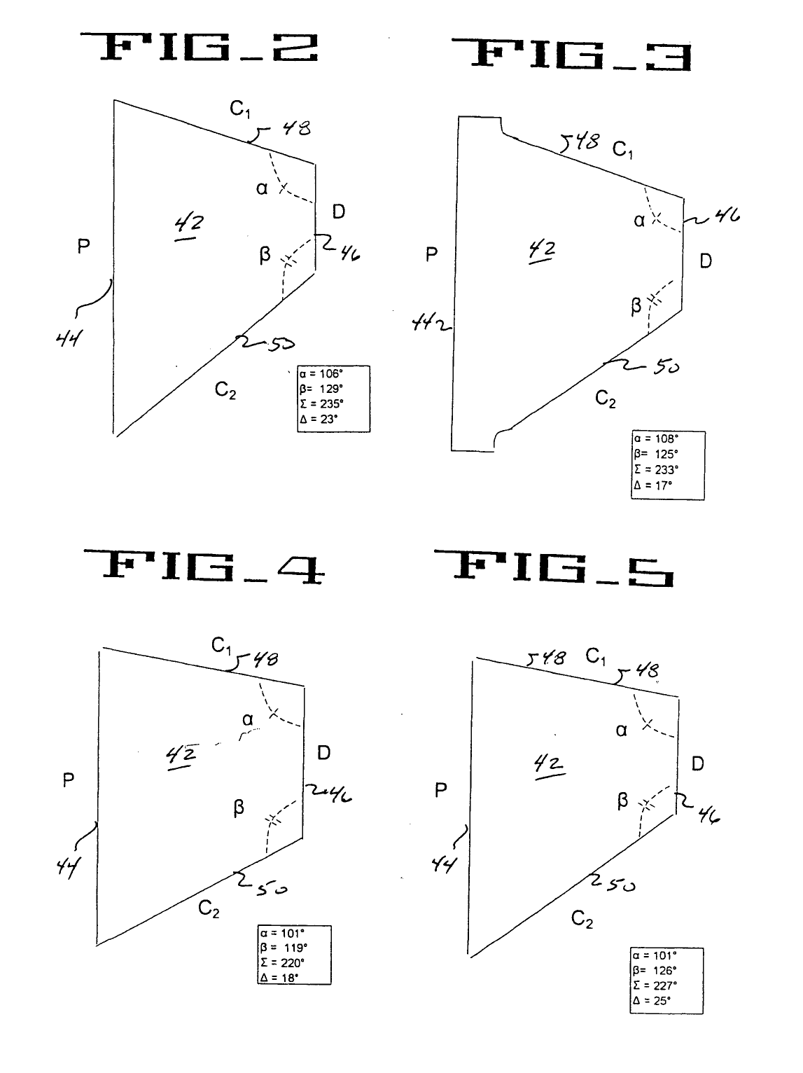

In accordance with the embodiment illustrated in FIG. 1, and as further illustrated in FIGS. 2 through 8, each of the elastomeric side panels 14, 16 includes an elastomeric substrate 42 which is specifically configured to enhance the fit of the present disposable absorbent garment. In particular, each of the elastomeric substrates 42 includes a proximal edge 44, a distal edge 46 to which fastening element 38 is joined, a first connecting edge 48, and a second connecting edge 50, with the first and second connecting edges 48, 50 extending between proximal and distal edges 44, 46.

As illustrated, the proximal edge 44 of each of the elastomeric substrates of side panels 14, 16 is joined to a respective one of the opposite marginal portions 26, 28 of the absorbent assembly 12 generally at the rear portion thereof.

In accordance with the present invention, elastomeric substrate 42 of each elastomeric side panel 14, 16, has been specifically configured to promote enhanced fit of the present disposable garment to a wearer, while at the same time promoting manufacture of the side panels from suitable elastomeric material while minimizing the creation of waste material. In particular, the proximal edge 44 and distal edge 46 of each elastomeric substrate are generally linear, and parallel to each other. In contrast, the first connecting edge 48 and second connecting edge 50, while being linear, are non-parallel to each other, and are non-perpendicular to the distal edge 46 of the elastomeric substrate 42.

With particular reference to FIGS. 2-8, illustrating various embodiments of the elastomeric substrate 42 of each elastomeric side panel, an angle α is defined between the first connecting edge 48 and the distal edge 46 of the elastomeric substrate, with the angle α being greater than 90°. In turn, an angle β is defined between the second connecting edge 50 and the distal edge 46 of the elastomeric substrate, with angle β being greater than 90°. In accordance with the present invention, angle β is greater than angle α.

In accordance with the present invention, the difference between angle α and angle β is preferably greater than about 15°, and preferably less than about 40°. It is presently preferred that the sum of angle α and angle β is less than about 260°, with the sum of angle α and angle β preferably being greater than about 220° and less than about 240°.

Specifically selected lengths for the elastomeric substrate 42 of the elastomeric side panels 14, 16 have been found to desirably enhance the comfort and fit of the present disposable absorbent garment. In particular, it is presently preferred that the proximal edge 44 of each elastomeric substrate 42 has a length between about 10.3 cm and about 20 cm, with the proximal edge 44 of each elastomeric substrate comprising about 20% to about 35% of the fully extended length of the disposable absorbent garment. It is further preferred that a ratio of the length of the proximal edge 44 to the distal edge 46 of each elastomeric substrate 42 be between about 2:1 and about 8:1.

It is presently preferred that angle α be greater than about 100°, preferably greater than about 110°, and more preferably greater than about 120°. In turn, it is preferred that angle β be greater than about 120°, preferably greater than about 130°, and more preferably greater than about 140°, again, with it being presently preferred that the sum of angle α and β be greater than about 220°, and less than about 240°.

Various configurations for each elastomeric substrate 42 are illustrated in FIGS. 2-8. In the embodiment of FIG. 2, angle α is illustrated as being 106°, and angle β as being 129°. In FIG. 3, wherein connecting edges 48, 50 are generally linear, and include stepped portions adjacent proximal edge 44 to facilitate connection to absorbent assembly 12, angle α is illustrated as being 108°, and angle β as being 125°.

In the embodiment of FIG. 4, angle α is illustrated as being 110°, and angle β as being 119°. In contrast, the embodiment of FIG. 5 also shows angle α as being 101°, while in this embodiment, angle β is illustrated as being 126°.

In the embodiment of FIG. 6, angle α is illustrated as being 100°, and angle β illustrated as being 136°.

Further preferred embodiments of the present invention are illustrated in FIGS. 7 and 8, wherein in FIG. 7, angle α is illustrated as being 123°, and angle β as being 139°, and in FIG. 8, angle α is illustrated as being 96°, and angle β as being 107°.

By the illustrated arrangements of the elastomeric side panels of the present invention, the provision of linear connecting edges arranged in specific relationship are believed to promote enhanced fit and comfort for the wearer of the present disposable absorbent garment. The tapered shape for each of the elastomeric side panels desirably acts to direct and maintain tension on the side panel to provide good fit through the waist portion and leg openings of the disposable garment, thereby enhancing fit of the garment about the legs and about the torso of a wearer. Additionally, the angle of the first connecting edge 48 desirably acts to help position the elastomeric side panel under the stomach of a wearer to minimize “roll-over” of this edge, and any rubbing or chaffing of the stomach area, thus enhancing comfort and fit.

Notably, these benefits, including the tension in the position of the first connecting edge 48 relative to the stomach of a wearer, and the disposition of the second connecting edge 50 adjacent to the leg of the wearer, allow for the present disposable absorbent garment to be raised or lowered without having to open the fastening system. This is sometimes referred to as a “convertible” arrangement, in that a diaper or like garment can be fitted to a wearer in an open condition, closed by securing the fastening element 38 of each of the elastomeric side panels 14, 16, with the garment thereafter being easily removed and refitted much in the nature of a conventional under pant or panty.

If desired, the present disposable absorbent garment can be configured generally in accordance with the teachings of U.S. Pat. No. 6,627,289, hereby incorporated by reference. Mechanical fastening arrangements for the present disposable absorbent garment can be configured in accordance with U.S. Pat. No. 5,053,028, No. 4,894,060, and No. 5,019,065, all hereby incorporated by reference.

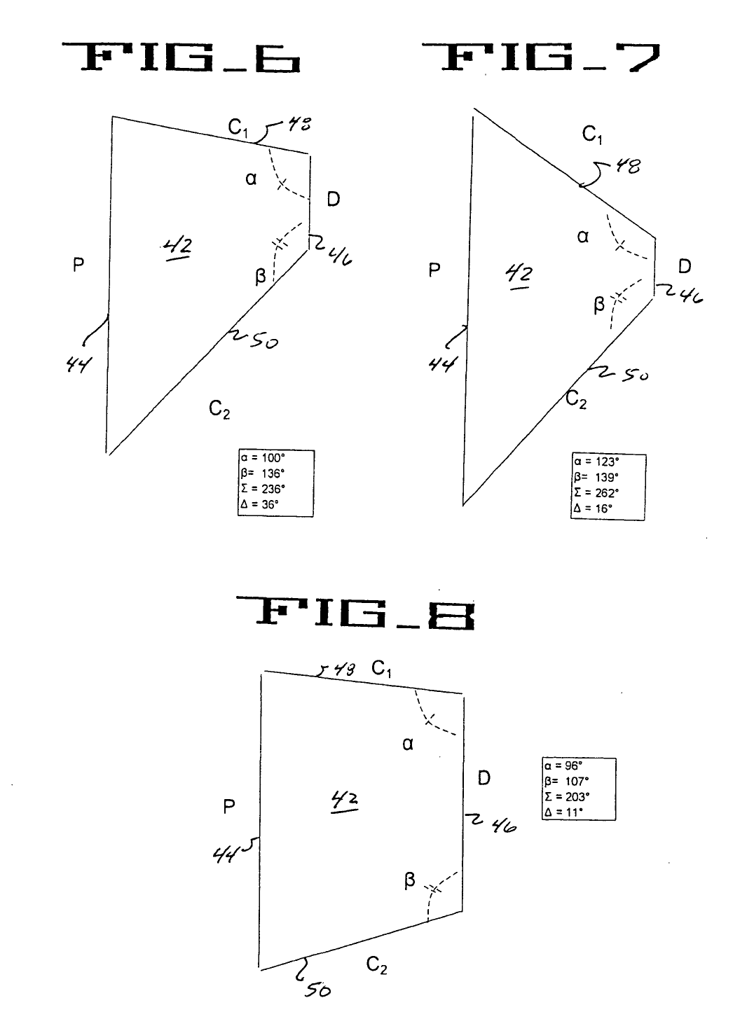

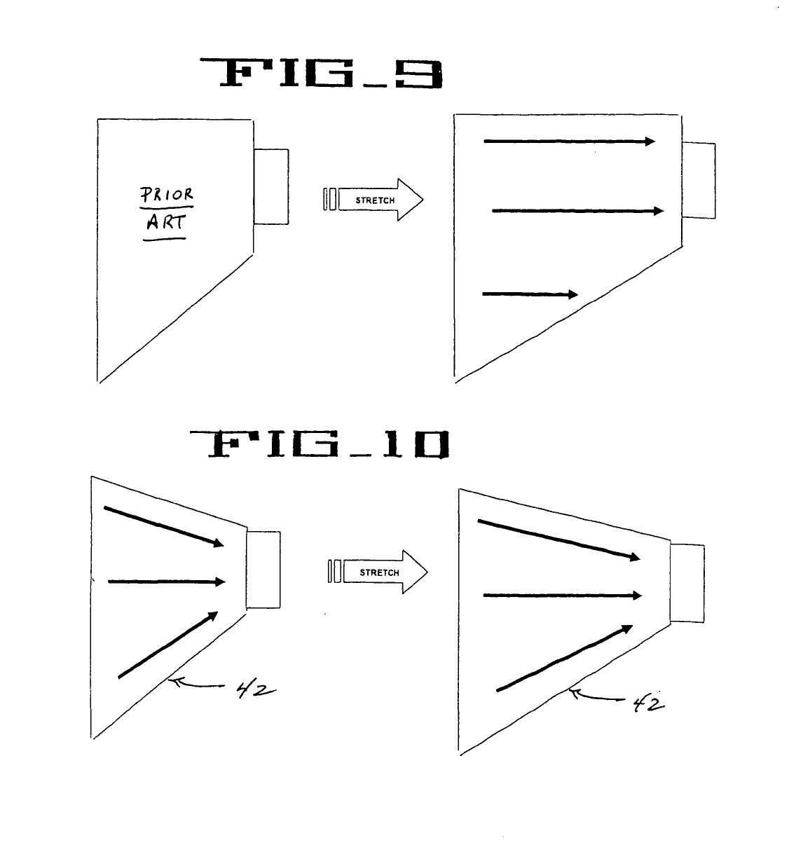

FIGS. 9 and 10 diagrammatically illustrate tensional forces generated within the elastomeric side panels of the present disposable absorbent garment (FIG. 10), and elastomeric side panels of the prior art. As will be observed, in the typical prior art arrangement illustrated in FIG. 9, tensional forces are directed generally parallel to each other. In contrast, a disposable absorbent garment including elastomeric side panels, including an elastomeric substrate 42 as illustrated in FIG. 10, desirably acts to shape and direct tensional forces in non-parallel relationship to each other, thus enhancing fit and comfort of the associated absorbent garment.

From the foregoing, it will be observed that numerous modifications and variations can be effected without departing from the spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed is:

1. A disposable absorbent garment, comprising:
an absorbent assembly having front and rear portions, and opposite marginal portions; and
a pair of elastomeric side panels configured to facilitate fitment of said absorbent garment to a wearer,
each side elastomeric side panel comprising an elastomeric substrate, and a fastening element,
each said elastomeric substrate comprising a proximal edge, a distal edge, and first and second connecting edges extending between said proximal and distal edges, said proximal edge being joined to a respective one of the opposite marginal portions of said absorbent assembly at the rear portion thereof, with the distal edge having the respective fastening element joined thereto for securing each elastomeric side panel in overlapping relationship with the front portion of said absorbent assembly,
said proximal edge and said distal edge of each said elastomeric substrate being linear and parallel to each other,
said first connecting edge and said second connecting edge of each said elastomeric substrate being linear and non-parallel to each other, and non-perpendicular to the distal edge of the elastomeric substrate,
wherein an angle α is defined between the first connecting edge and the distal edge of each elastomeric substrate, angle α being greater than 90°, and an angle β is defined between the second connecting edge and the distal edge of each elastomeric substrate, angle β being greater than 90° and greater than angle α.

2. A disposable absorbent garment in accordance with claim 1, wherein:
the difference between angle α is greater than about 15°.

3. A disposable absorbent garment in accordance with claim 2, wherein:
the difference between angle α and angle β is less than about 40°.

4. A disposable absorbent garment in accordance with claim 1, wherein:
the sum of angle α and angle β is less than 260°.

5. A disposable absorbent garment in accordance with claim 1, wherein:
angle α is greater than about 100°, and angle β is greater than about 120°.

6. A disposable absorbent garment in accordance with claim 5, wherein:
angle α is greater than about 110°, and angle β is greater than about 130°.

7. A disposable absorbent garment in accordance with claim 6, wherein:
angle α is greater than about 120°, and angle β is greater than about 140°.

8. A disposable absorbent garment in accordance with claim 1, wherein:
said proximal edge of each said elastomeric substrate has a length between about 10.3 cm and about 20 cm.

9. A disposable absorbent garment in accordance with claim 8, wherein:
said proximal edge of each said elastomeric substrate comprises about 20% to 35% of a length of said disposable absorbent garment.

10. A disposable absorbent garment in accordance with claim 1, wherein:
a ratio of the length of said proximal edge to said distal edge of each elastomeric substrate is between about 2:1 and about 8:1.

11. A disposable absorbent garment, comprising:
an absorbent assembly comprising an absorbent matrix, and having front and rear portions, and opposite side marginal portions; and
a pair of elastomeric side panels configured to facilitate fitment of said absorbent garment to a wearer,
each said elastomeric side panel comprising an elastomeric substrate, and a mechanical fastening element,
each said elastomeric substrate comprising a proximal edge, a distal edge, and first and second connecting edges extending between said proximal and distal edges, said proximal edge being joined to a respective one of the opposite marginal portions of the absorbent assembly at the rear portion thereof, with the distal edge of each elastomeric substrate having the respective mechanical fastening element joined thereto for securing the elastomeric side panel in overlapping relationship with the front portion of the absorbent assembly,
said proximal edge and said distal edge of each said elastomeric substrate being linear and parallel to each other,
said first connecting edge and said second connecting edge of said elastomeric substrate being linear and non-parallel to each other, and non-perpendicular to the distal edge of the elastomeric substrate,
wherein an angle α is defined between the first connecting edge and the distal edge of each elastomeric substrate, angle α being greater than 90°, and an angle β is defined between the second connecting edge and the distal edge of each elastomeric substrate, angle β being greater than 90°, with angle β being greater than angle α,
wherein a sum of angle α and angle β is less than 260°, and the difference between angle α and angle β is greater than about 15°, and less than about 40°.

12. A disposable absorbent garment in accordance with claim 11, wherein:
the sum of angle α and angle β is greater than about 220° and less than about 240°.

13. A disposable absorbent garment in accordance with claim 11, wherein:
said proximal edge of each said elastomeric substrate has a length between about 10.3 cm and about 20 cm, wherein said proximal edge of each said elastomeric substrate comprises about 20% to 35% of a length of said disposable absorbent garment.

14. A disposable absorbent garment in accordance with claim 11, wherein:
said mechanical fastening element comprises a hook fastening element.

15. A disposable absorbent garment in accordance with claim 13, wherein a ratio of the length of said proximal edge to said distal edge of each elastomeric substrate is betwen about 2:1 and about 8:1.
